(19) United States
(12) Patent Application Publication
(10) Pub. No.: US 2008/0210223 A1
(43) Pub. Date: Sep. 4, 2008

(54) Apparatus and Method for Repositioning Abdominal Fatty Tissue

(75) Inventors: Sharon Joines, Raleigh, NC (US); Jessica Stahl, Winston-Salem, NC (US); Glenwood Morris, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: North Carolina State University

(21) Appl. No.: 11/948,295

(22) Filed: Nov. 30, 2007

Related U.S. Application Data
(60) Provisional application No. 60/861,947, filed on Nov. 30, 2006.

(51) Int. Cl. A61F 5/24

(52) U.S. Cl. 128/96.1

(57) ABSTRACT

The present invention is directed to an apparatus and associated method for repositioning fat tissue in a patient's abdominal region during medical and surgical procedures. In particular, the apparatus of the present invention is particularly adapted for repositioning an abdominal pannus and exposing the lower abdominal and groin regions of a patient. In one embodiment, the present invention includes a base member and a support member that angularly rotatable with respect to the base member. The support member includes a surface that contacts the pannus of a patient and pushes it superiorly away from the groin region.

### CROSS REFERENCE TO RELATED APPLICATIONS

This application claims the benefit of Provisional Application No. 60/861,947, filed on Nov. 30, 2006, the contents of which are hereby incorporated by reference.

### FIELD OF THE INVENTION

The present invention relates generally to the field of medical and surgical devices, and more particularly to device and system for repositioning fat tissue in a patient's abdominal region during a medical or surgical procedure.

### BACKGROUND OF THE INVENTION

Increasingly, obesity is becoming a significant health concern in the United States. In addition to the health risks inherent with obesity, the size and shape of the human body can have adverse effects on the ability of health care workers to adequately treat obese patients. In particular, some overweight or obese patients may have developed what is known as an abdominal pannus or apron of fat tissue.

A pannus is a region of loose skin and fatty tissue in the lower abdomen that tends to descend below the abdomen, appearing as an apron. In some cases, a pannus can completely obscure the patient's lower abdominal regions and groin area. As a result, the pannus may make it difficult for health workers to provide needed medical care to such patients. For example, the presence of a pannus can make it difficult for health care workers to perform certain procedures, such as gynecological and urological procedures, insertion and removal of catheters, diagnostic imaging procedures, changing dressings, etc.

Currently, there exists no adequate method of repositioning the pannus, and most procedures are limited to simple and ineffectual remedies. For example, one such remedy requires two to workers to pull the pannus away from an area of interest using a towel or sheet while a third health care worker provides care to the patient. Other procedures of repositioning the pannus may include using tape or some other adhesive to lift it out of the groin region. These methods have generally proven unreliable, as the skin on the underside of a pannus is typically not sufficiently clean or rigid to maintain its form under the stress of being restrained with such methods. As a result, the pannus may not be adequately restrained and in some cases may be released to its resting place over the groin area. Further, these procedures can be uncomfortable for the patient, and may also put the health care workers at risk for injury.

Accordingly, there exists a need for an apparatus or system that can be used by a single health care worker to reposition the pannus to permit the health care worker to provide the needed care, while minimizing discomfort to the patient.

### BRIEF SUMMARY OF THE INVENTION

The present invention is directed to an apparatus and associated method for repositioning fat tissue in a patient's abdominal region during medical and surgical procedures. In particular, the apparatus of the present invention is particularly adapted for repositioning an abdominal pannus and exposing the lower abdominal and groin area of a patient. In one embodiment, the present invention includes a base member and a support member that angularly rotatable with respect to the base member. The support member includes a surface that contacts the pannus of a patient and pushes it forward away from the groin region.

In one embodiment, the support member includes a central portion having a surface for repositioning the pannus and a pair of legs that are adapted to be received by a pair of corresponding receiving members disposed on the base member. The receiving members cooperate with legs so that the position of the support member with the respect to the base member is angularly adjustable. For example, the support member can initially be positioned so that the central portion of the support member is positioned towards the patient's legs adjacent to the groin region. In this initial position, a portion of the support member can be positioned between the pannus and groin region of the patient. The support member can then be rotated upwardly so that the central portion of the support member is moved generally in the direction of the patient's upper torso. As a result, the pannus is repositioned so that a health care worker can gain access to otherwise poorly accessible areas of the patient's body, such as the lower abdominal area and groin region.

In some embodiments, the base member can include a plurality of pairs of receiving members that are disposed on opposite sides of the base member and that comprise oblong openings having varying widths with respect to each other. In this embodiment, the receiving members each comprise an oblong opening formed in the base member through which at least a portion of each leg is insertable. Since pairs of receiving members have varying widths and the sides of the legs of the support member are tapered towards the distal portions of the legs, the support member can be inserted into each of the pairs of receiving members to a different extent. As a result, a health care worker can select an appropriate pair of receiving members based on a physical characteristic of the patient, such as weight or girth. In a further embodiment, the legs can be retracted or inserted out of or inserted into the support member to thereby accommodate patients of varying weight and girth.

Accordingly, the apparatus of the present invention can be used to perform procedures on a wide variety of patients of varying size and shape. Further, the support member is typically constructed of a lightweight material that can be relatively easily position and rotated within the base member. As a result, the present invention provides an apparatus that can be used by a single person and which also helps to eliminate discomfort that may otherwise be experienced by the patient and caregiver.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a perspective view of an apparatus for repositioning fat tissue away from the groin region in the process of being used on a patient;

FIG. 2 is a perspective view of the apparatus of FIG. 1;

FIGS. 3A-3C are cross-sectional side views depicting the process of the support member being rotatably moved in a receiving member;

FIGS. 4A-4B illustrate a process of using the apparatus of FIG. 1 that is in accordance with one embodiment of the invention;

FIG. 5 is a perspective view of an alternative embodiment of an apparatus for repositioning fat tissue away from the groin region;

FIG. 6. is a partial view of the support member of the apparatus of FIG. 1 depicting that the leg of the support member can be retracted out of and inserted into the support member;

FIGS. 7A-7C is a side view of a receiving member of the apparatus depicted in FIG. 5, depicting the receiving member rotating between various positions; and

FIGS. 8A-8B depict an embodiment of the invention in which the support member includes fastening mechanisms for attaching sanitary materials to the support member.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

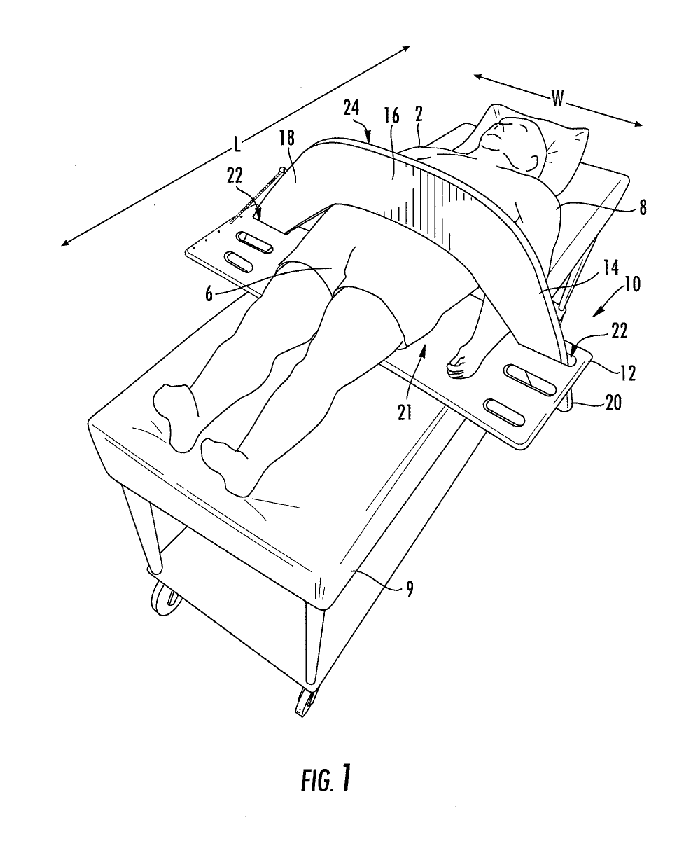

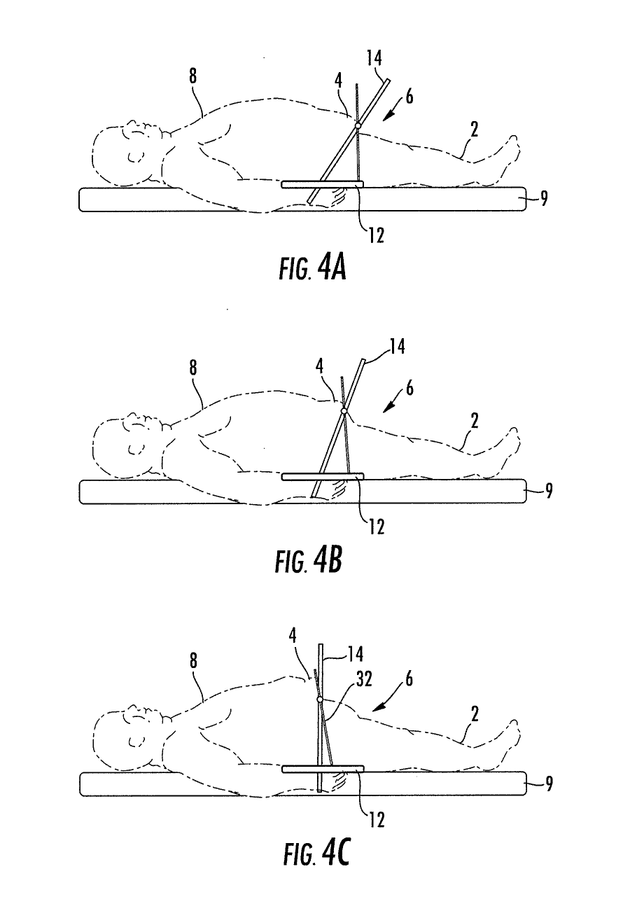

The present invention is directed to a device and associated method that can be used to effectively reposition a portion of a patient's fat tissue in the abdominal region to help provide improved access to the lower abdominal and groin regions. With reference to FIG. 1, an apparatus 10 for repositioning fat tissue in a patient's abdominal region is illustrated in use on a patient 2. For purposes of the following discussion, the patient is depicted as having a series of regions generally designated as an abdominal apron or pannus (see briefly, FIG. 4A, reference character 4), a groin region or lower abdominal region 6, and an upper torso 8.

As shown in FIG. 1, the patient 2 is laying in a prone position on a table 9, which should be understood to represent an operating or examination table as commonly known in the medical fields. In the context of the present discussion, the lateral direction extends along the lateral width of the table, designated generally by the reference character W, and the longitudinal direction extends along the longitudinal length of the table, designated generally by the reference character L.

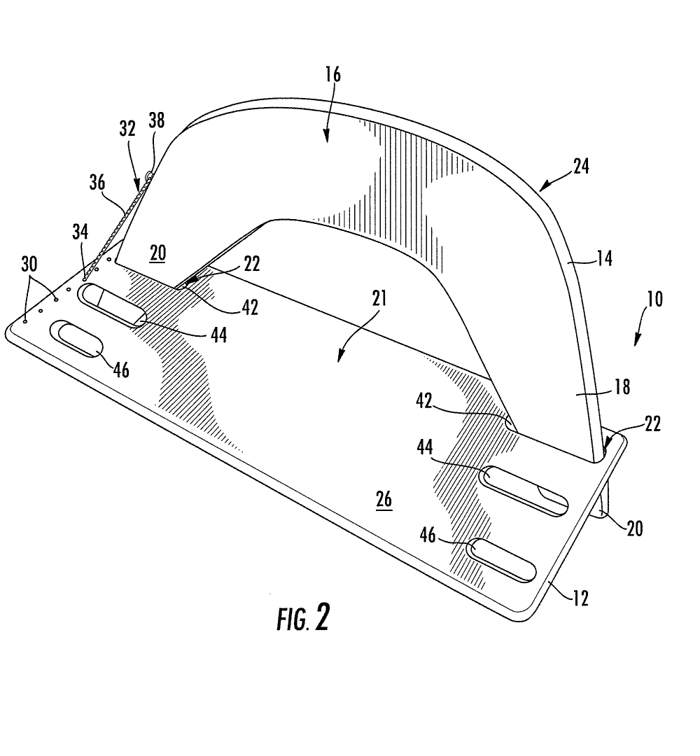

As can best be seen in FIGS. 1 and 2, the apparatus 10 includes a base member 12 which is disposed below the patient and a support member 14 that is positioned above the patient near the patients groin or lower abdominal region. The support member includes a central portion 16 and a pair of legs 18 disposed towards opposite sides of the central portion. Preferably, the legs extend downwardly from the central portion and are tapered so that the width of the legs narrows towards the distal portion 20 of each leg. The base member 12 is disposed beneath the patient 2 and spans the width of the table as well as the patient. The base member includes a central region 21 that underlies the patient. The base member also includes at least one pair of receiving members 22 that are disposed on opposite sides of the base member and the patient. For example, in FIG. 1, the patient is depicted as being disposed between the receiving members. The receiving members 22 are each configured and arranged to cooperatively receive a distal portion 20 of one of the legs 18 of the support member 14. In this way, the apparatus 10 is disposed about the patient with the support member being positioned above the patient near the groin or abdominal region 6.

The receiving members 22 cooperate with legs 18 so that the position of the support member with the respect to the base member is angularly adjustable. For example, the support member can initially be positioned so that the central portion of the support member is positioned towards the patient's legs (see briefly FIG. 4A). In this initial position, a portion of the support member can be positioned between the pannus and groin region of the patient. The support member can then be rotated upwardly so that the central portion of the support member is moved generally in the direction of the patient's upper torso (see briefly FIG. 4B). The central portion 16 of the support member also includes a rigid or semi-rigid front surface 24 that extend laterally across the patient's mid-section and that is configured to engage at least a portion of the patient's pannus 4. Rotation of the support member from the initial position towards the patient's upper torso causes surface 24 to push the pannus away from the groin region superiorly towards the patient's upper torso 8. As a result, the pannus is repositioned so that a health care worker can gain access to areas of the patient's body, such as the groin region.

As can best be seen in FIG. 2, the apparatus includes a locking member that is configured and arranged to secure and maintain the support member in a desired position with respect to the base member. In the illustrated embodiment, the locking member comprises a plurality of spaced-apart apertures 30 that extend longitudinally along the length of the base member and a corresponding shaft 32 that is attached to the support member. The apertures are configured and arranged to receive a distal portion 34 of the shaft therein to thereby brace the support member in a desired position with respect to the base member. As shown, the shaft includes a plurality of notches 36 that are capable of gripping an edge of the upper surface 26 of the base member. As shown, the support member includes a clamp 38 that can be used to secure the shaft to the support member. The clamp 38 can be used to alter a length of the shaft that extends in the direction of the apertures to help facilitate securing of the support member in different positions with respect to the base member.

In an alternative embodiment, the locking member can comprise a shaft that is not attached to the support member. In this embodiment, the shaft includes a distal portion having a first diameter that is small enough to be inserted into the apertures and a second and larger diameter that is larger than the diameter of the apertures. As a result, a portion of the shaft extends upwardly from the aperture and braces the back surface of the support member in a desired position. Generally, the spacing of the apertures can be selected based on the desired level of control over the position of the support member. For example, more tightly spaced apertures allow a greater degree of control over the position of the support member. It should be recognized that a wide variety of different types of locking members can be used to maintain the support member in a desired position with respect to the base member including clamps, hooks, mechanical fasteners, loops, wedges, blocks, and the like.

In one embodiment, the receiving members comprise a pair of oblong openings disposed on opposite sides of the base member through which each corresponding leg of the support member is insertable. In this embodiment, the distal portions of the legs are capable of pivotal movement within each receiving member in the longitudinal direction of the base member. As a result, the position of the support member is angularly adjustable with respect to the base member as discussed above.

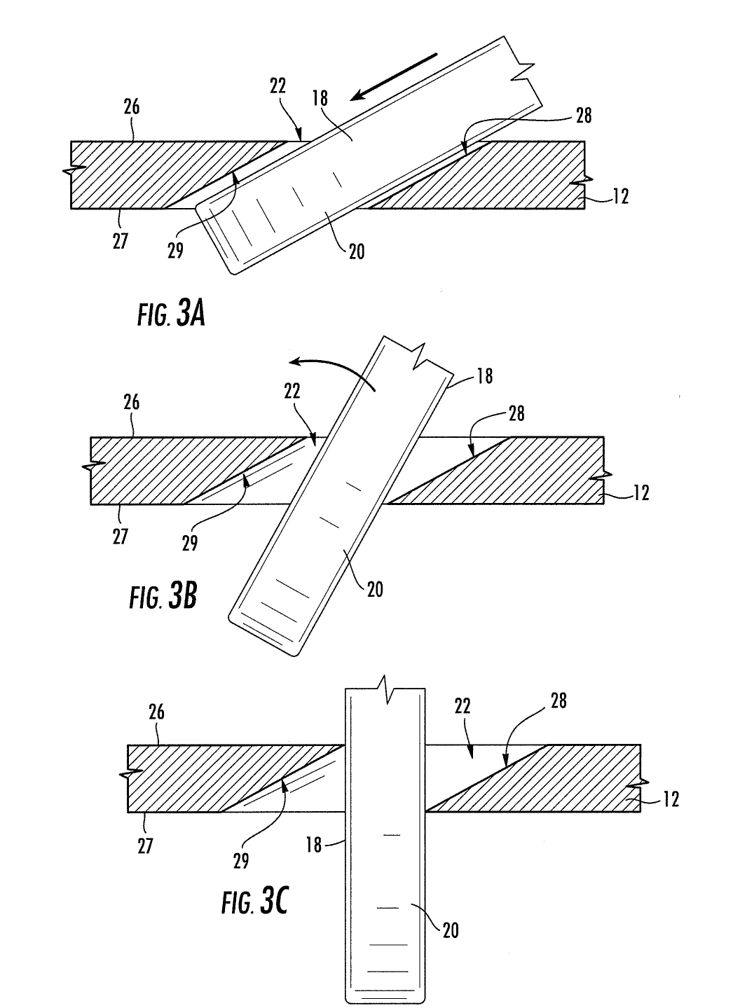

In some embodiments, the oblong openings (e.g., receiving members) may include chamfered edges that permit the distal portions of the legs to be pivotably rotated within the receiving members so that the support member can in turn be positioned at various angles with respect to the base member. In this regard, FIGS. 3A-3C illustrate a distal portion 20 of one of the legs 18 of the support member being inserted into a corresponding receiving member 22 and being rotated upwardly. As shown the, upper and lowers surfaces 26, 27 of the base member 12 are chamfered to form beveled surfaces 28, 29, respectively. As shown, the beveled surfaces are disposed opposite each other in the oblong openings and help facilitate rotation of the legs within the receiving members so that the support member can be rotated upwardly in the direction of the patient's upper torso. The beveled surfaces 28, 29 are angled away from the surfaces 28, 29 of the base member. For example, the angle between bevel surface 28 and upper surface 26 typically ranges from about 20 to 80 degrees, with a range between 30 and 60 degrees being preferred. In the illustrated embodiment, the angle between beveled surface 28 and upper surface 26 is about 45 degrees. Similarly, the angle between bevel surface 29 and lower surface 27 typically ranges from about 20 to 80 degrees, with a range between 30 and 60 degrees being preferred. In the illustrated embodiment, the angle between beveled surface 29 and lower surface 27 is about 45 degrees. Preferably, beveled surfaces 28, 29 are parallel to each other to help facilitate insertion and rotation of the support member within the receiving members.

In the embodiment depicted in FIGS. 1 and 2, the base member includes a plurality of pairs of receiving members having openings with a generally oblong shape. As shown, the plurality of pairs of receiving members extend longitudinally along the length of the base member from the patient groin region towards the upper torso. As shown, each successive pair of receiving members (e.g., oblong openings) has a width (e.g., in the lateral direction) that is greater than the width of the preceding pair of receiving members. For example, receiving members 46 have a width that is greater than the preceding pair of receiving members 44. Accordingly, the legs of the support member positioned in receiving members 46 will be able to be inserted further into the receiving members 46 than would be possible in receiving members 44. As a result, the height of the support member with respect to the base member can be selected to accommodate patients of varying sizes and dimensions. In particular, a health care worker can select a pair of receiving members in which to insert the legs of the support member based on a physical characteristic of the patient, such as girth or weight. For examples, receiving members 42 can be selected to accommodate a smaller person, such as from about 250 to 450 lbs, receiving members 44 can be selected to accommodate a medium obese person, such as from about 450 to 550 lbs, and receiving members 46 can be selected to accommodate a large obese person, such as greater than 550 lbs.

Desirably, the inner portion of the support member that is closest to the patient has an elliptical or curved shape that approximates the contours of the human body. For example, in the illustrated embodiment, the portion of the support member facing the patient forms a generally hemispherical arc that can be positioned around the patient. In this way, it is possible for front surface 24 to maximize contact with the pannus of the patient.

In the embodiments illustrated in FIGS. 1 and 2, the support member comprises a unitary piece that can be relatively easily sterilized for use with multiple patients. Ideally, the apparatus 10 is composed of a synthetic material or plastic that is durable so that it can be sterilized and used repeatedly. Further, the apparatus is preferably non-radio opaque such that it will not interfere with any radiological procedures that the patient must undergo while his or her abdomen is being restrained. Suitable materials for the support member as well as the base member include nylon, polyester and polyethylene, such as high density polyethylene. Other materials may be used although not necessarily with equivalent results.

FIGS. 4A-4C illustrate a method of using the apparatus 10 that is in accordance with the invention. As shown, a patient is positioned on table 9 with the base member 12 disposed between patient 2 and the table 9. Based on the size of the patient, the base member is positioned so that the appropriate pair of receiving members are aligned with a desired location of the patient's body. The support member 14 is inserted into the receiving member at an angle that permits the front surface of the support member to engage and contact the pannus 4 of the patient. Typically, the initial position of the support member is selected so that the top of the support member is directed towards the lower portion of the patient's body.

Once the legs of the support member have been inserted into the receiving members, the health care worker can then push the support member towards the upper torso 8 of the patient. Ideally, the support member is inserted to a depth that allows the front surface of the support member to contact and reposition a large proportion of the patient's pannus. As shown in FIG. 4B, rotation of the support member in the direction of the upper torso 8 causes the pannus 4 to be repositioned away from the groin region 6. When a desired amount of the pannus has been repositioned, the support member can be maintained in a desired position with shaft 32.

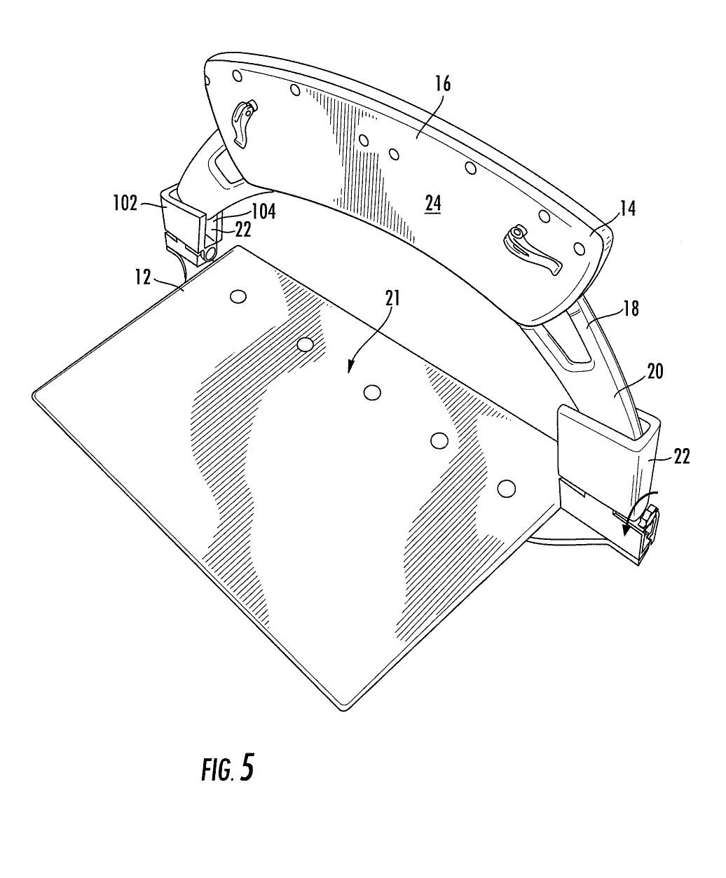

With reference to FIG. 5, an alternative embodiment of an apparatus for repositioning fat tissue in a patient's abdominal region is illustrated and broadly designated by reference number 100. In this embodiment, the apparatus 100 includes base member 12 and support member 14. Support member 14 includes a central portion 16 have front surface 24 and legs 18 disposed towards opposite ends of the central portion. The base member includes a pair of receiving members 22 disposed on opposite sides of the base member 12. The receiving members include side walls 102 that together define a space 104 therebetween for receiving the legs 18 of the support member. As discussed in greater detail below, the receiving members 22 are capable of pivoting so that the position of the support member is angularly adjustable with respect to the base member.

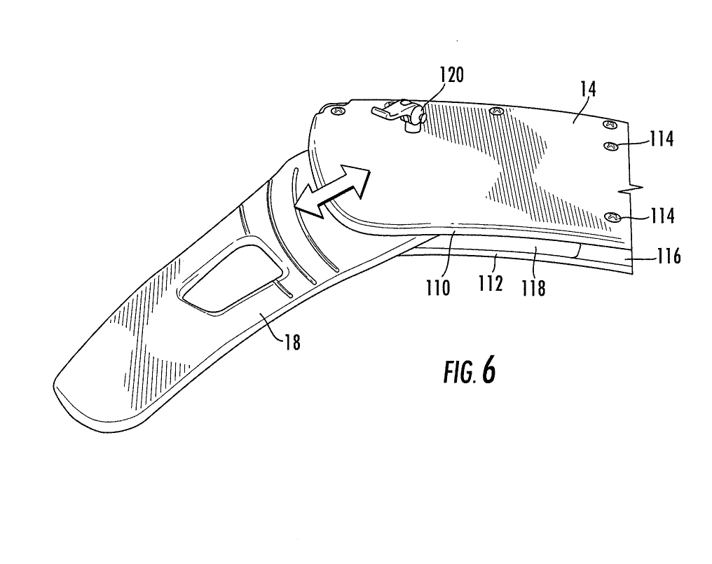

To accommodate patients of varying girth and weight, the legs of the support member can be controllably retracted into the support member. This allows the height of the support member with respect to the base member to be adjusted as needed. In this regard, FIG. 6 depicts that the legs can be moved in to and out of the support member. In this embodiment, the support member includes a multi-piece construction in which the support member comprises first and second pieces 110, 112, that are attached to each other with one or more fasteners 114, such as bolts or screws. A spacer piece 116 is positioned between first and second pieces 110, 112 so that the first and second pieces 110, 112 are disposed in a spaced apart relation with each other. As a result, space 118 exists between the first and second pieces. Space 118 is configured and arranged to permit the legs of the support member to be retracted therefrom and inserted therein.

In the illustrated embodiment, the support member includes a clamping member 120 that is capable of pulling the first and second pieces towards each other in order to maintain the legs in a desired position. For example, the clamping member squeezes the first and second pieces of the support member together to frictionally secure the legs in a desired state of extension.

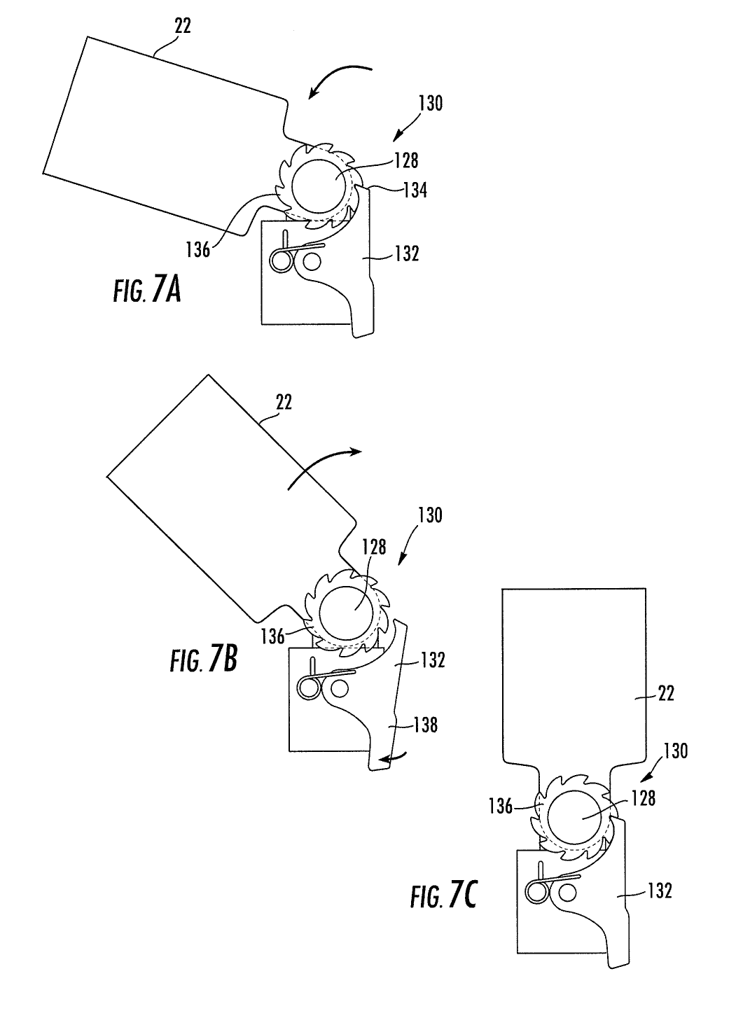

As briefly noted above, receiving members 22 are able to pivotably rotate to thereby rotate the support member into a desired position with the base member. As shown in FIGS. 7A-7C, one of the receiving member is depicted in the process of rotating from a generally prone position (FIG. 7A) to an upright position (FIG. 7B). Generally, each receiving member independently includes a shaft 128 which facilitates rotation of the receiving. In this embodiment, at least one of the receiving members includes a locking member for maintaining the support member in a desired position. For example, FIGS. 7A-7C illustrate a receiving member having a ratchet-like locking member 130. The locking member 130 includes a spring biased pawl 132 having a proximal end 134 that is configured to engage a spur gear 136 disposed towards the base of the receiving member on shaft. Engagement of the proximal end of the pawl with the spur gear prevents rotation of the shaft. As shown in FIG. 7B, the locking member can be disengaged by applying pressure to a distal portion 138 of the pawl 132. It should be recognized that the above described locking mechanism represents one method that can be used to maintain the support member in a desired position, and that other methods can be used such as those discussed above.

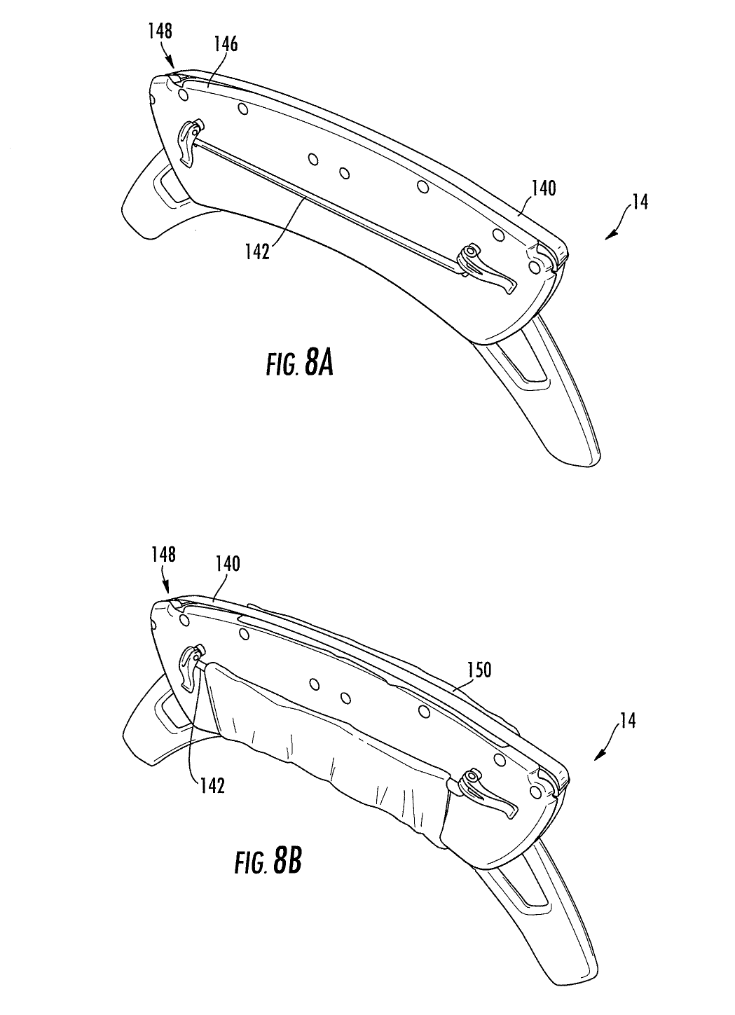

In some embodiments, it may be desirable to attach a sanitary material, such as a chuck, to the support member. In this regard, FIGS. 8A and 8B illustrate an embodiment of the invention in which the support member includes fastening mechanisms for attaching a sanitary cloth to the support members. As shown, the support member includes a tensioning belt 140 and a strap 142 that can be used to secure a sanitary cloth to the support member. The tensioning belt 140 extends across a top portion 146 of the support member and includes a clamping mechanism 148 that can be used to apply tension and tighten the belt 140. Preferably, the strap 142 comprises a flexible elastomeric material, such as bungee cord material, that biases the cloth towards the surface of the support member. As shown in FIG. 8B, a sanitary cloth 150 is secured under the tensioning belt and wraps around the strap 142 so that the position of the cloth is maintained during the procedure.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. An apparatus for repositioning fat tissue in a patient's abdominal region, the apparatus comprising:
a support member having a central portion and a pair of legs disposed on opposite sides of the central portion, said central portion having a surface configured to extend laterally across a patient's torso;
a base member having a central region upon which a patient can be positioned, the base member including at least one pair of receiving members disposed on opposite sides of the base member, said receiving members each being configured to releasably receive a corresponding distal portion of one of the legs of the support member therein, wherein the support member is configured to be angularly adjustable with respect to the base member so that upward angular movement of the support member away from said base member causes said surface of the support member to engage and reposition a portion of a patient's abdominal fat tissue; and
a locking member for maintaining the support member in a desired angular position with respect to the base member.

2. The apparatus of claim 1, wherein the receiving members each comprise an oblong opening formed in the base member through which at least a portion of each leg is insertable.

3. The apparatus of claim 2, wherein each leg is configured to pivot within a corresponding receiving member.

4. The apparatus of claim 2, wherein the base member includes a plurality of pairs of receiving members that extend longitudinally along a length of the base member, and in which each pair of receiving member has a width that is different than the other pairs of receiving members.

5. The apparatus of claim 1, wherein the support member comprises a unitary structure comprising a sterilizable plastic material.

6. The apparatus of claim 1, wherein the support member comprises a material selected from the group consisting of high density polyethylene, nylon, and polyester.

7. The apparatus of claim 1, wherein an inner portion of the support member has a generally hemispherical arc-like shape that approximates the contour of a human body.

8. The apparatus of claim 1, wherein the receiving members are each rotatably disposed about a shaft.

9. The apparatus of claim 8, wherein the support member includes an interior space from which the pair of legs are extendable.

10. An apparatus for repositioning fat tissue in a patient's abdominal region, the apparatus comprising:
a base member having a central region upon which a patient can be positioned, the base member having a plurality of pairs of receiving members that extend longitudinally along a length of the base member, and in which each pair of receiving member comprises an oblong opening having a width that is different than the other pairs of receiving members;
a cooperating support member that is configured to be positioned above the base member, the support member having a pair of legs that are adapted to be received by a pair of said receiving members, the support member including a central portion disposed between the pair of legs and having a rigid or semi-rigid surface that is configured to engage and reposition a portion of a patient's abdominal fat tissue away from the patient's groin region.

11. The apparatus of claim 10, wherein the base member is positioned on a table.

12. The apparatus of claim 10, wherein each of the oblong openings includes a pair of opposing beveled surfaces that are configured to facilitate upward rotation of the legs within the receiving members.

13. The apparatus of claim 10, wherein the support member comprises a unitary structure comprising a sterilizable plastic material selected from the group consisting of high density polyethylene, nylon, and polyester.

14. The apparatus of claim 10, further comprising a locking member for maintaining the support member in a desired angular position with respect to the base member

15. A method of repositioning the pannus of a patient comprising:
positioning a base member on a table, the base member having at least one pair of receiving members disposed on opposite sides of the base member;
positioning a patient in a prone position on the table so that the base member is located towards the patients abdominal region, wherein the patient includes a pannus;
providing a support member having a central portion and a pair of legs disposed on opposite sides of the central portion;
inserting the pair of legs in a corresponding pair of receiving members so that a front surface of the support member contacts the pannus region of the patient;
rotating the support member upwardly towards an upper torso of the patient such that the pannus is repositioned towards the patient's upper torso to thereby expose areas of the patient's groin region.

16. The method of claim 15, wherein the base member includes a plurality of pairs of receiving members that extend longitudinally along a length of the base member, and in which each pair of receiving member comprises an oblong opening a width that is different than the other pairs of receiving members.

17. The method of claim 16, further comprising the step of selecting a pair of receiving members in which to insert the legs of the support member based on a physical characteristic of the patient.

18. The method of claim 15, further comprising the step of locking the support member in a position after the pannus has been repositioned.

19. The method of claim 15, wherein the front surface of the support member is initially disposed at least partially between the pannus and a groin region of the patient.
